(19) United States
(12) Patent Application Publication
(10) Pub. No.: US 2020/0107716 A1
(43) Pub. Date: Apr. 9, 2020

(54) APPARATUS FOR COMPREHENSIVE MULTI-SENSORY SCREENING AND METHODS THEREFOR

(71) Applicant: IVISION TECHNOLOGIES, LLC, Des Plaines, IL (US)

(72) Inventors: Stuart Paul Richer, Highland Park, IL (US); Robert Endo, Northbrook, IL (US); Douglas K. Oshana, Lincolnwood, IL (US)

(73) Assignee: IVISION TECHNOLOGIES, LLC, Des Plaines, IL (US)

(21) Appl. No.: 16/563,449

(22) Filed: Sep. 6, 2019

Related U.S. Application Data
(60) Provisional application No. 62/728,037, filed on Sep. 6, 2018.

(51) Int. Cl. A61B 3/00; A61B 3/18; A61B 5/12; G01M 11/02

(52) U.S. Cl. CPC A61B 3/0033; A61B 3/0008; A61B 3/005; G01J 3/524; A61B 5/125; G01M 11/0235; A61B 3/185

(57) ABSTRACT

In an embodiment, the invention provides an apparatus for comprehensive vision and audio screening, the apparatus including a housing having left and right separated compartments. Each of the left and right separated compartments contains a distance- and contrast-adjustable component of a stereo display that can be switched to be transparent with a rear display providing background and see-through capability, a semitransparent mirror, a lens with built-in camera, an LED module, a light source with a set of filters, a light module, and a component of a stereo speaker. The apparatus further includes a communication module, an accelerometer, a three-axis gyroscope, a light detector, a processor; a memory storing firmware, and a power supply component having a rechargeable battery and a charging component.

This application is a non-provisional of and claims the benefit of U.S. Provisional Patent Application No. 62/728,037 filed Sep. 6, 2018, the entire disclosure of which is incorporated herein by reference. The disclosures of U.S. Provisional Patent Application No. 62/728,044 filed Sep. 6, 2018 and U.S. Provisional Patent Application No. 62/728,039 filed Sep. 6, 2018 are also incorporated herein by reference in their entirety.

This application includes material which is subject to copyright protection. The copyright owner has no objection to the facsimile reproduction by anyone of the patent disclosure, as it appears in the Patent and Trademark Office files or records, but otherwise reserves all copyright rights whatsoever.

### FIELD

The present invention relates in general to the field of screening devices and methods for vision and auditory screening.

### SUMMARY

In general, example embodiments of the present invention provide an improved system and method of vision, hearing, cognition and proprioception testing as the key features of the system. The disclosed system and method facilitate greater efficiency and throughput of patient flow, and enable contemporary achievements in video and communication technologies.

### BRIEF DESCRIPTION OF THE DRAWINGS

The foregoing and other objects, features, and advantages of the invention will be apparent from the following more particular description of preferred embodiments as illustrated in the accompanying drawings, in which reference characters refer to the same parts throughout the various views. The drawings are not necessarily to scale, emphasis instead being placed upon illustrating principles of the invention.

FIG. 1A shows a graphical view illustrating a process in accordance with an embodiment of the invention.

FIG. 1B shows a perspective view illustrating a headwear test unit (HWTU).

FIG. 2 shows a block diagram illustrating configuration and operation of the HWTU in accordance with an embodiment of the invention.

FIG. 2A shows a block diagram illustrating configuration and operation of the HWTU in accordance with a further embodiment.

FIG. 2B shows a block diagram illustrating operation of the HWTU in accordance with yet a further embodiment.

FIG. 2C shows a front elevational view of an HWTU housing.

FIG. 3 shows a block diagram illustrating configuration and operation of a camera in accordance with an embodiment of the invention.

FIG. 4 shows a block diagram illustrating configuration and operation of first and second cameras in accordance with an embodiment of the invention.

FIG. 5 shows an operational block diagram illustrating operation of a processor controller component.

### DETAILED DESCRIPTION

Reference will now be made in detail to the preferred embodiments of the present invention, examples of which are illustrated in the accompanying drawings. The following description and drawings are illustrative and are not to be construed as limiting. Numerous specific details are described to provide a thorough understanding. However, in certain instances, well-known or conventional details are not described in order to avoid obscuring the description. References to one or an embodiment in the present disclosure are not necessarily references to the same embodiment; and, such references mean at least one.

Reference in this specification to “an embodiment” or “the embodiment” means that a particular feature, structure, or characteristic described in connection with the embodiment is included in at least an embodiment of the disclosure. The appearances of the phrase “in an embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not other embodiments.

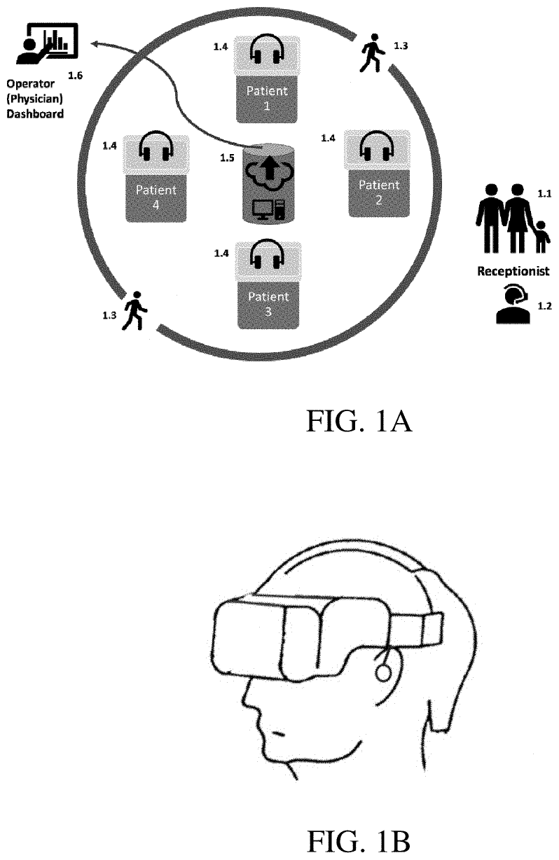

With reference to FIG. 1A, in accordance with an embodiment of the invention, a patient (1.1) checks in with receptionist (1.2) and then sits down in one of secure patient chairs (1.4) and lifts the test device attached to a central service column. The test device is configured to turn on and off by lifting the headwear unit from the cradle. According to some embodiments, a circular sound-proof partition is provided with two entrances/exits (1.3). An arc of approximately ninety degrees is provided for each patient to use hand gestures. In an embodiment, the central “service column” (1.5) provides the necessary service hardware, including at least one processor, charging station, lift cradles, test devices (a headwear test unit, HWTU, FIG. 1B), wired and wireless communication means. The information collected from the test devices is communicated to the operator dashboard (1.6). As illustrated in FIG. 1B, in an embodiment, the invention provides a head-mounted online or offline examination machine and belongs to the technical field of vision testing, audio testing, and optometry.

An object of the present invention is to provide a headwear test unit (HWTU) (FIG. 1B) and corresponding methods intended for comprehensive multisensory screening including but not limited to vision, hearing and cognitive patient screening.

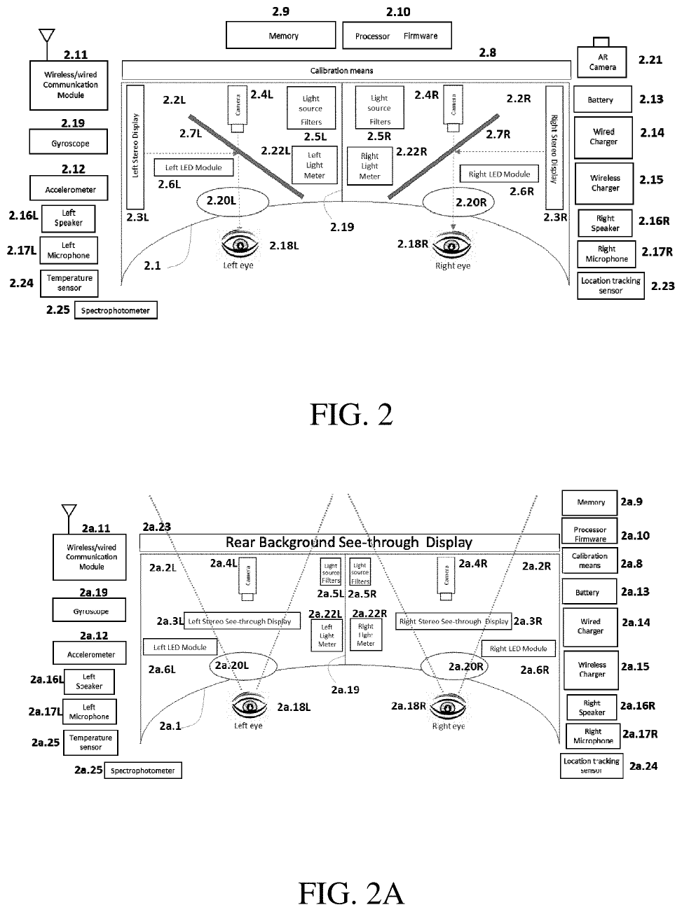

The headwear test unit block diagram (HWTU) in accordance with an embodiment thereof is shown in FIG. 2. The unit includes:
a housing 2.1 having two compartments 2.2L and 2.2R;
a compartment divider 2.19;
semitransparent mirrors 2.7L and 2.7R;
LED modules 2.6L and 2.6R;
light source and filters 2.5L and 2.5R;
stereo displays 2.3L and 2.3R;
processor and the corresponding firmware 2.10;
built-in memory 2.9;
eye monitoring and iris recognition cameras 2.4L and 2.4R featuring optional autofocus functionality to get perfect picture of the eye; with high enough frame rate to detect and measure blinking;
a pair of lenses 2.20L and 2.20R, which could be optionally removable form the vision path;
close-to-the-ear stereo speakers 2.16L and 2.16R that can be ear mounted, next to the ear, direction speakers away from the ear, or use bone conduction.
stereo microphones 2.17L and 2.17R;
a wireless and wired communication module communicating with but not limited to the central “service column” (FIG. 1, 1.5);
rechargeable battery 2.13;
the wired and wireless charging modules 2.14 and 2.15 accordingly;
the calibration means 2.8;
the accelerometer 2.12 and gyroscope 2.19; and
the augmented reality (AR) front looking camera (2.21)
light meters (2.22L and 2.22R)
temperature sensor (2.24)
location tracking sensor (2.23)
spectrophotometer sensor (2.25)
single or multiple proximity sensors, and
mechanical and/or electrical adjustments to calibrate horizontal and vertical alignment

The eye monitoring and iris recognition cameras are configured for photographing still images and capturing video.

The ear-mounted stereo speakers and stereo microphones are used for hearing screening as well as for audio communication between the system and a patient.

In some embodiments, the HWTU is divided into left and right compartments, which are fixed to the housing on the left and right side, respectively.

Some embodiments have hygienic material bonded to their face and/or fixed to the rear end edge of the housing, the hygienic material being either replaceable per patient or cleanable with antibacterial properties.

In some embodiments, a processor controls the test sequences, display of test images, and audio signal generation, and performs control functions during the unit calibration, as well as the information post processing.

Further, the semitransparent mirrors are intended to independently reflect the stereo display images to the patient's eyes as well, allowing taking of still images and video recording of the eyes with said cameras. In another embodiment, the front mounted camera of the HWTU is used by low vision/visually impaired subjects to magnify, brighten and filter images.

In some embodiments, calibration components are provided. Such calibration components may include a proximity sensor to identify the correct HWTU position and the light sensors.

In some embodiments, said accelerometer is used to capture the patient input provided by patient's head movement.

In yet another embodiment, the HWTU has a fixed or soft cursor and a patient moves their head until a cursor is over a selection. Then a patient employs a selection device to lock said selection. The selection device can be a clicker, a prolonged blink, or any other means of confirmation.

In an embodiment, the three-axis gyroscope is used for identification of a patient's head position and for monitoring.

Yet another HWTU embodiment is shown in FIG. 2A. This embodiment includes left and right stereo see-through displays (2a.2L and 2a.3R) and rear background see-through display 23 in FIG. 2A. However, this embodiment does not necessarily include the left and right semitransparent mirrors. The main differences between the embodiment of FIG. 2A and other embodiments are that cameras are situated behind the stereo displays and the display needs to be put in the transparent mode for the camera to take photographs of the eye. In addition, the rear display can provide black color as a background for the stereo display and block the light from the inside of the HWTU. When both stereo displays and rear display are put in transparent mode, the patient is able to see their surroundings, with either or both of the stereo displays providing augmented reality information and images.

Yet another HWTU implementation does not have the left and right semitransparent mirrors (FIG. 2b). Displays and cameras are separated horizontally or vertically so as not to interfere with one another and to allow the patient to see the displays, and to allow for camera to take a picture of the eye.

In some embodiments (FIG. 2c), the HWTU housing comprises two movable compartments 2c.2 and 2c.3 where the horizontal and vertical movements are controlled by dials 2c.5 and 2c.6 (left compartment) and 2c.7 and 2c.8 (right compartment).

Each side's optics and display is setup independently so they can be adjusted laterally/horizontally to match interpupillary distance (IPD).

Said embodiment also gives independence to move both sides vertically to center both sides to match pupil's position. Movement can be done by electric motor or manually.

In another embodiment, the invention provides a single horizontal and single vertical adjustment for both compartments.

Alternatively, the HWTU can be configured with a hinge 2c.4 and the IPD adjustment can be performed by movement around said hinge in addition to independent horizontal and vertical adjustments.

Further, some embodiments comprise mechanical or electrical means 2c.6L and 2c.6R to bring in and take away lenses 2c.5L and 2c.5R from the view line.

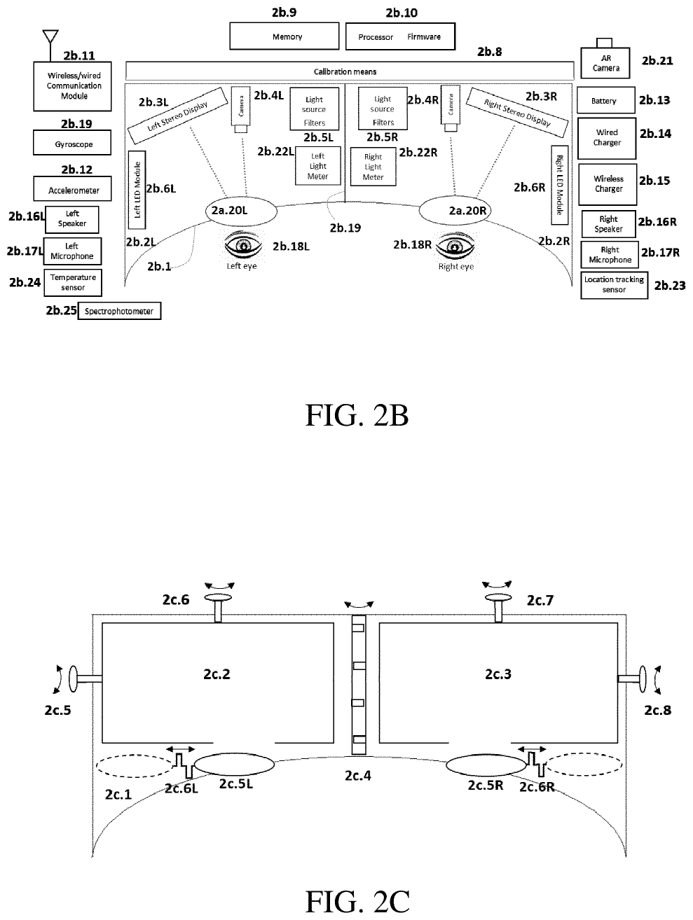

FIG. 3 shows a detailed diagram depicting the “display-semitransparent mirror-LED module-camera-patient's eye” ray traces.

In some embodiments, the camera is configured to take a photograph of an eye through a custom lens that has a portion at the bottom or the top that has no magnification (FIG. 3, Front view).

In some embodiments, the HWTU includes means for providing other degrees of freedom for calibrating the system for a particular patient. Such means include but are not limited to: display adjustment, lens lateral adjustment, mirror tilting, camera tilting and autofocus, image shifting, and liner light bending.

In various embodiments, the present invention can have the following significant advantages. The present apparatus can be used for (but is not limited to) a comprehensive combined hearing, cognition and vision examination. The present apparatus supports authentication and authorization procedures. The present apparatus can be used for said comprehensive screening at a patient's home and can be configured to communicate the screening results to the system secure server for post processing via Internet using Wi-Fi or cellular communication means.

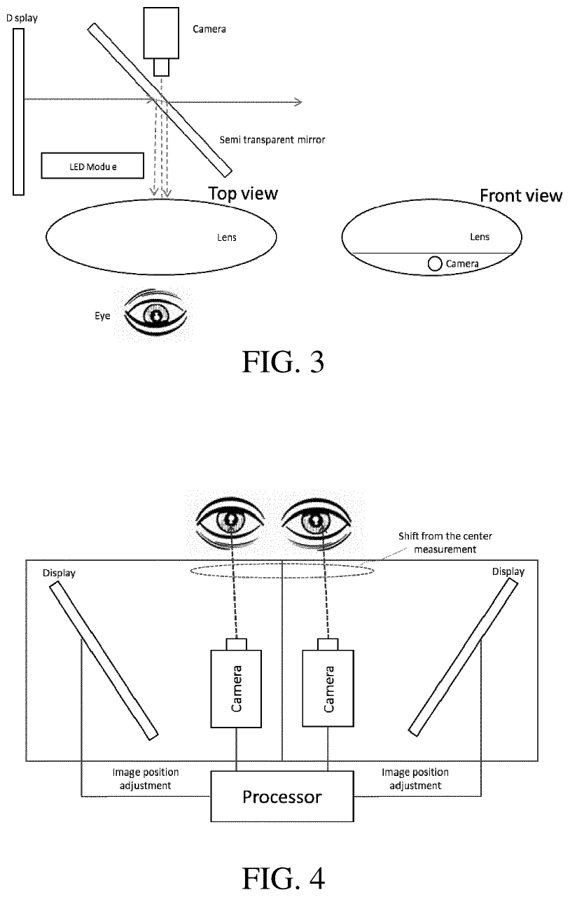

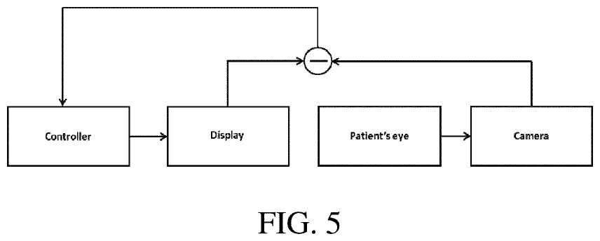

According to some embodiments, the automatic closed-loop control system can be implemented to allow positioning of the display image according to a particular patient's interpupillary distance (IPD). See FIG. 4 and FIG. 5.

The image position adjustment signal is calculated by the processor controller component and is based on a pre-defined correlation between the patient's IPD (As measured with two cameras) and the default image position. See FIG. 5.

The user input sub-system is intended for standardized auditory and visual combined instructions to improve understanding of the test and conduct the testing routine more efficiently, which can be particularly important for handicapped and older patients. In some embodiments said sub-system comprises four or more patient response options to support user input:
IR hand motion
IR/BR laser pointer with the laser and single button
voice recognition
mouth pointer (i.e. for quadriplegic)
four button hand controller
head motion
foot tapping

The user input subsystem can have, but is not limited to, the following user input taxonomy:
Alpha
Binary
Numeric
Directional—Up/Down, Left/Right
Directional—N, NE, E, SE, S, SW, W, NW
Sliding Bar
Shape
Object
Color

In some embodiments, the invention includes a six-direction keypad in the shape of, for example, a six-pointed star. The six-direction keypad can be activated to provide the user input discussed above.

In an embodiment, the invention provides an apparatus for comprehensive vision and audio screening, the apparatus comprising of a full set or a subset of components below:
a housing having left and right separated compartments where said left (right) compartment contains respectively:
   a left (right) distance and contrast adjustable component of a stereo display that can optionally be transparent with rear display providing background and see through capability
   a left (right) semitransparent mirror
   a left (right) lens with built-in camera
   a left (right) LED module
   a light source with a set of filters
   a left (right) light module
   a left (right) component of a stereo microphone
   a left (right) component of a stereo speaker
a wired communication module;
a wireless communication module;
an accelerometer;
a three-axis gyroscope;
a light detector;
a proximity sensor;
a processor;
a memory storing firmware;
a calibration compartment;
a temperature sensor;
a location tracking sensor;
a spectrophotometer sensor; and,
a power supply component having a rechargeable battery and a charging component.

In an embodiment, the invention provides an apparatus that provides a closed loop system for automatic image position adjustment on the display based on the inter pupil distance (IPD).

In an embodiment, the invention provides an apparatus that provides manual or guided position adjustment display based on the interpupillary distance (IPD).

In an embodiment, the invention provides an apparatus that includes a forward-looking camera to provide AR capabilities to the patient and improves spatial awareness, particularly for hand gestures.

In an embodiment, the invention provides an apparatus that provides a per-person calibration means supporting one or more of the functions consisting of: display adjustment, lens lateral adjustment, mirror tilting, camera tilting, autofocus, image shifting.

In an embodiment, the invention includes an apparatus that provides measurements, verification and confirmation that a patient can fit the system parameters based on IPD and iris focus.

In an embodiment, the invention includes an apparatus that provides a mechanism and method to bring in and take away lenses from the view line.

In an embodiment, the invention provides an apparatus that includes a spectrophotometer to measure spectral characteristics of eyeglasses which is built into HWTU or into external fixture.

In an embodiment, the invention provides an apparatus that includes components and algorithm to capture opacification of the human lens with each eye after dilation of the eyes by the doctor.

The present invention is described above with reference to block diagrams and operational illustrations of methods and devices to provide comprehensive multisensory screening. It is understood that each block of the block diagrams or operational illustrations, and combinations of blocks in the block diagrams or operational illustrations, may be implemented by means of analog or digital hardware and computer program instructions. These computer program instructions may be stored on computer-readable media and provided to a processor of a general-purpose computer, special purpose computer, ASIC, or other programmable data processing apparatus, such that the instructions, which execute via the processor of the computer or other programmable data processing apparatus, implements the functions/acts specified in the block diagrams or operational block or blocks. In some alternate implementations, the functions/acts noted in the blocks may occur out of the order noted in the operational illustrations. For example, two blocks shown in succession may in fact be executed substantially concurrently or the blocks may sometimes be executed in the reverse order, depending upon the functionality/acts involved.

At least some aspects disclosed can be embodied, at least in part, in software. That is, the techniques may be carried out in a special-purpose or general-purpose computer system or other data processing system in response to its processor, such as a microprocessor, executing sequences of instructions contained in a memory, such as ROM, volatile RAM, non-volatile memory, cache or a remote storage device. Functions expressed in the claims may be performed by a processor in combination with memory storing code and should not be interpreted as means-plus-function limitations.

Routines executed to implement the embodiments may be implemented as part of an operating system, firmware, ROM, middleware, service delivery platform, SDK (Software Development Kit) component, web services, or other specific application, component, program, object, module or sequence of instructions referred to as “computer programs.” Invocation interfaces to these routines can be exposed to a software development community as an API (Application Programming Interface). The computer programs typically comprise one or more instructions set at various times in various memory and storage devices in a computer, and that, when read and executed by one or more processors in a computer, cause the computer to perform operations necessary to execute elements involving the various aspects.

A machine-readable medium can be used to store software and data which when executed by a data processing system causes the system to perform various methods. The executable software and data may be stored in various places including for example ROM, volatile RAM, non-volatile memory and/or cache. Portions of this software and/or data may be stored in any one of these storage devices. Further, the data and instructions can be obtained from centralized servers or peer-to-peer networks. Different portions of the data and instructions can be obtained from different centralized servers and/or peer-to-peer networks at different times and in different communication sessions or in a same communication session. The data and instructions can be obtained in entirety prior to the execution of the applications. Alternatively, portions of the data and instructions can be obtained dynamically, just in time, when needed for execution. Thus, it is not required that the data and instructions be on a machine-readable medium in entirety at a particular instance of time.

Examples of computer-readable media include but are not limited to recordable and non-recordable type media such as volatile and non-volatile memory devices, read only memory (ROM), random access memory (RAM), flash memory devices, removable disks, magnetic disk storage media, optical storage media (e.g., Compact Disk Read-Only Memory (CD ROMS), Digital Versatile Disks (DVDs), etc.), among others.

In general, a machine-readable medium includes any mechanism that provides (e.g., stores) information in a form accessible by a machine (e.g., a computer, network device, personal digital assistant, manufacturing tool, any device with a set of one or more processors, etc.).

In various embodiments, hardwired circuitry may be used in combination with software instructions to implement the techniques. Thus, the techniques are neither limited to any specific combination of hardware circuitry and software nor to any particular source for the instructions executed by the data processing system.

As used herein, and especially within the claims, ordinal terms such as first and second are not intended, in and of themselves, to imply sequence, time or uniqueness, but rather are used to distinguish one claimed construct from another. In some uses where the context dictates, these terms may imply that the first and second are unique. For example, where an event occurs at a first time, and another event occurs at a second time, there is no intended implication that the first time occurs before the second time. However, where the further limitation that the second time is after the first time is presented in the claim, the context would require reading the first time and the second time to be unique times. Similarly, where the context so dictates or permits, ordinal terms are intended to be broadly construed so that the two identified claim constructs can be of the same characteristic or of different characteristic.

While some embodiments can be implemented in fully functioning computers and computer systems, various embodiments are capable of being distributed as a computing product in a variety of forms and are capable of being applied regardless of the particular type of machine or computer-readable media used to actually effect the distribution.

The above embodiments and preferences are illustrative of the present invention. It is neither necessary, nor intended for this patent to outline or define every possible combination or embodiment. The inventors have disclosed sufficient information to permit one skilled in the art to practice at least one embodiment of the invention. The above description and drawings are merely illustrative of the present invention and that changes in components, structure and procedure are possible without departing from the scope of the present invention as defined in the following claims. For example, elements and/or steps described above and/or in the following claims in a particular order may be practiced in a different order without departing from the invention. Thus, while the invention has been particularly shown and described with reference to embodiments thereof, it will be understood by those skilled in the art that various changes in form and details may be made therein without departing from the spirit and scope of the invention.

What is claimed is:

1. An apparatus for comprehensive vision and audio screening, the apparatus comprising:
a housing having left and right separated compartments;
said left compartment containing:
a left distance- and contrast-adjustable component of a stereo display that can be switched to be transparent with a rear display providing background and see-through capability;
a left semitransparent mirror;
a left lens with built-in camera;
a left LED module;
a left light source with a first set of filters;
a left light module; and,
a left component of a stereo speaker;
said right compartment containing:
a right distance- and contrast-adjustable component of a stereo display that can be switched to be transparent with the rear display providing background and see-through capability;
a right semitransparent mirror;
a right lens with built-in camera;
a right LED module;
a right light source with a second set of filters;
a right light module; and,
a right component of a stereo speaker;
at least one communication module;
an accelerometer;
a three-axis gyroscope;
a light detector;
a processor;
a memory storing firmware; and,
a power supply component having a rechargeable battery and a charging component.

2. The apparatus for comprehensive vision and audio screening in accordance with claim 1, wherein the apparatus further comprises a spectrophotometer sensor.

3. The apparatus for comprehensive vision and audio screening in accordance with claim 1, wherein the apparatus further comprises a calibration compartment.

4. The apparatus for comprehensive vision and audio screening in accordance with claim 1, wherein the apparatus further comprises a temperature sensor.

5. The apparatus for comprehensive vision and audio screening in accordance with claim 1, wherein the apparatus further comprises a location tracking sensor.

6. The apparatus for comprehensive vision and audio screening in accordance with claim 1, wherein the apparatus further comprises a proximity sensor.

7. The apparatus for comprehensive vision and audio screening in accordance with claim 1, wherein the left compartment contains a left component of a stereo microphone and the right compartment contains a right component of the stereo microphone.

8. The apparatus for comprehensive vision and audio screening in accordance with claim 1, further comprising a closed loop system for automatic image position adjustment on the display based on the interpupillary distance.

9. The apparatus for comprehensive vision and audio screening in accordance with claim 1, further comprising a forward-looking camera to provide AR capabilities to the patient and improve spatial awareness.

10. The apparatus for comprehensive vision and audio screening in accordance with claim 1, further comprising a per-person calibration means supporting one or more of the functions consisting of: display adjustment, lens lateral adjustment, mirror tilting, camera tilting, autofocus, image shifting.

11. The apparatus for comprehensive vision and audio screening in accordance with claim 1, further comprising a mechanism configured to move lenses inward and outward from a view line.

12. The apparatus for comprehensive vision and audio screening in accordance with claim 1, further comprising

[symbol]

13. The apparatus for comprehensive vision and audio screening in accordance with claim 1, further comprising

[symbol]

14. The apparatus for comprehensive vision and audio screening in accordance with claim 1, further comprising a spectrophotometer configured to measure spectral characteristics of eyeglasses, the spectrophotometer being built into the headwear test unit or into a fixture external to the headwear test unit.

15. An apparatus calibration system, the system comprising:
a headwear test unit having a plurality of cameras, displays, and LEDs;
a light intensity detector facing said displays and LEDs, the light intensity detector being operable to run a self-calibration test by measuring and adjusting intensity of said displays and LEDs;
a charging station;
a spectrometer configured to measure spectral characteristics of a patient's eye glasses;
at least one calibrated camera facing displays and LED such that it can be used to measure color accuracy, contrast accuracy and other aspects of the displays and LEDs.

16. The apparatus calibration system in accordance with claim 8, wherein the charging station has a form factor that blocks light coming into the head unit.

17. The apparatus calibration system in accordance with claim 8, wherein the intensity detector is configured to measure light intensity during a plurality of vision tests and provide feedback to the headwear test unit that enables the headwear test unit to perform adjustments.

18. The apparatus calibration system in accordance with claim 8, wherein said at least one calibrated camera is part of the headwear test unit, part of the charging unit, or part of a calibration unit/stand.

19. The apparatus calibration system according to claim 8, wherein the light intensity detector is operable to periodically run said self-calibration test.

20. The apparatus calibration system according to claim 8, wherein the light intensity detector is built into a charging station and is operable to run said self-calibration test each time the headwear test unit is charged.
